(19) United States
(12) Patent Application Publication
(10) Pub. No.: US 2012/0225309 A1
(43) Pub. Date: Sep. 6, 2012

(54) Method of Preparing a Polyetherimide Coating on a Metallic Substrate

(75) Inventors: Tapan Kumar Rout, Odisha (IN); Anil Vilas Gaikwad, De Haarlem (NL); Theo Dingemans, Leiden (NL); Magali Audrey Valerie Wainer, Ac Haarlem (NL)

(73) Assignee: TATA STEEL NEDERLAND TECHNOLOGY BV, Ijmuiden (NL)

(21) Appl. No.: 13/497,419

(22) PCT Filed: Sep. 24, 2010
(86) PCT No.: PCT/EP2010/005848

§ 371 Date: Mar. 21, 2012

(30) Foreign Application Priority Data
Sep. 24, 2009 (EP) 09012146.8
Dec. 18, 2009 (EP) 09015691.0

(51) Int. Cl. C08G 73/10; B32B 15/08; B05D 3/02; C07C 229/26; B05D 7/14; B05D 3/00

(52) U.S. Cl. 428/458; 427/388.1; 562/565; 528/310

(57) ABSTRACT

A method of preparing a polyetherimide coating on a carbon steel substrate, which includes the steps of: providing an organic solvent, providing a dianhydride, providing a first diamine that is a monoaromatic diamine, providing a second diamine, placing the organic solvent, the dianhydride, the first diamine and the second diamine in a reaction vessel to form a reaction mixture, stirring the reaction mixture under inert conditions to form a polyamic acid intermediate, applying the polyamic acid intermediate on the carbon steel substrate, and curing the polyamic acid intermediate to form a polyetherimide coating.

The present invention relates to a polyetherimide coated carbon steel substrate, a polyamic acid intermediate and a method for preparing the said polyetherimide carbon steel coated substrate and the said polyamic acid intermediate. The invention further relates to a method for preparing a polyetherimide fibre from the polyamic acid intermediate.

Metal bodies such as rebars, wires, strips or sheets are subjected to protective surface treatments to prolong the longevity and performance of the rebar, wire, strip or sheet. This operation is of particular importance in the automotive industry and white good industries where the metal sheet should exhibit very high corrosion resistance.

Zinc coatings have been used in the past since they provide a continuous impervious metallic barrier that does not allow moisture to contact the metal. Without direct moisture contact corrosion should not occur. However, zinc coatings gradually degrade over time due to exposure to water and atmospheric pollutants in open air applications. Barrier life is also proportional to coating thickness and thicker coatings increase the costs of coating metal substrates with zinc.

Zinc coatings are also oxidised preferentially when bare metal is exposed to moisture if the metal is scratched. In the immediate presence of zinc, iron in a metal is not oxidised until all of the zinc has been sacrificed. However, the products of zinc oxidation have a high surface area and produce blisters that adversely affect the appearance of the covering paintwork.

In view of the above, organic coatings have been manufactured to improve corrosion resistance and reduce costs when coating metal substrates. It has been found that coating thickness is an important parameter that can affect coating performance. For instance, thicker coatings increase corrosion resistance but reduce sheet weldability and coating formability.

Organic coatings based on epoxy resins such as diglycidylether bisphenol A (DGEBA) and its derivatives have been deposited directly on metal substrates as corrosion resistant coatings. This process also involved creating a thin oxide film on the metal surface, which was intended to increase adhesion between the coating and the substrate. Despite the presence of the oxide film, coating adhesion was unsatisfactory due to reactive oxirane (—CH₂—CH—O⁻) groups characteristic of the epoxy largely disappearing upon curing. As a consequence of the poor adhesion a reduction in corrosion resistance was observed.

Organic coatings based on aromatic polyetherimides exhibit excellent adhesion and high temperature resistance, which has prompted their use as high performance materials in the electronics and aerospace sectors. Polyetherimides of this type are usually characterised by high glass transition temperatures (Tg) and high decomposition temperatures due to the presence of ordered aromatic groups along the polymeric backbone. As a consequence these polymers are difficult to process because they are only soluble in high boiling point protic solvents and at elevated temperatures. Such solvents comprise m-cresol or halogenated solvents such as tetrachloroethylene, many of which are toxic and not used in large industrial scale processes.

Some of these polyetherimides are semi-crystalline, rigid and poor in formability and subjecting such polyetherimide coatings to a forming operation is likely to induce a reduction in coating integrity and corrosion resistance due to crack formation.

It is therefore evident that an organic coating is needed that has better characteristics than those previously known.

It is an object of the invention to provide a polyetherimide coated substrate, wherein the polyetherimide coating has improved corrosion resistance.

It is an object of the invention to provide a polyetherimide coated substrate, wherein the polyetherimide coating has improved coating adhesion.

It is an object of the invention to provide a polyetherimide coated substrate, wherein the polyetherimide coating has improved formability.

According to a first aspect of the invention there is provided a method of preparing a polyetherimide coating on a carbon steel substrate, which comprises the steps of:
i. providing an organic solvent;
ii. providing a dianhydride;
iii. providing a first diamine that is a monoaromatic diamine;
iv. providing a second diamine;
v. placing the organic solvent, the dianhydride, the first diamine and the second diamine in a reaction vessel to form a reaction mixture;
vi. stirring the reaction mixture under inert conditions to form a polyamic acid intermediate;
vii. applying the polyamic acid intermediate on the carbon steel substrate;
viii. curing the polyamic acid intermediate to form a polyetherimide coating.

Polyetherimides prepared in accordance with the invention comprise a dianhydride, a first diamine and a second diamine. The dianhydride contains rigid aromatic groups that provide the polyetherimide with stiffness, improved corrosion resistance and improved mechanical properties. However, polyetherimide coatings, which comprise dianhydrides of the type described above are typically semi-crystalline and have insufficient flexibility and formability. It is therefore necessary to copolymerise the dianhydride with the first diamine and the second diamine to provide an amorphous polyetherimide coating having improved corrosion resistance, flexibility and formability.

According to a second aspect of the invention, polyetherimides having improved corrosion resistance, mechanical properties, flexibility and formability can also be prepared if step iii. of the above process is omitted and a second diamine having increased flexibility along its polymeric backbone is used. According to this process the polyamic acid intermediate and the polyetherimide coating comprise a dianhydride and a second diamine. Preferably, the second diamine comprises flexible ether linkages along its polymeric backbone and more preferably the second diamine is an aliphatic Jeff amine, an aromatic Jeff amine or a derivative thereof.

The step of applying the polyamic acid intermediate on the substrate before curing is advantageous since it permits the use of milder solvents that allow the polyamic acid intermediate to be applied on a substrate at room temperature. If the polyamic acid intermediate was first cured to form the polyetherimide then the application of the polyetherimide on the substrate would require elevated temperatures and the use of toxic high boiling point protic solvents. The invention therefore offers a significant advantage in terms of processability.

Preferably the polyetherimide coating comprises a third diamine that is a polyetherdiamine and preferably an aromatic polyetherdiamine since the aromatic groups contribute to improving corrosion resistance and the ether groups contribute to improving adhesion and the formability of the polyetherimide. The presence of ether groups can also improve coating adhesion since oxygen groups act as electron donating Lewis base sites. A non limiting example of a suitable aromatic polyetherimide is 4,4′-(1,3-Phenylenedioxy)dianiline (M1).

Preferably the polyetherimide coating comprises a first diamine that is a substituted monoaromatic diamine and preferably a meta substituted monoaromatic diamine since meta substituted diamine compounds disrupt intermolecular interactions that lead to mesophase behaviour and a reduction in coating flexibility and formability.

Preferably the polyetherimide coating comprises a first diamine that is m-phenylenediamine (MPA). The copolymerisation of the dianhydride, MPA and the second diamine results in an amorphous polyetherimide structure, which is highly flexible and highly formable. The amorphous nature of the coating is largely due to MPA inducing irregularities in the polymer chains morphology. The presence of MPA also has a positive effect with respect to the thermo-viscous behaviour of the polyamic acid intermediate which should lead to improved flexibility and flow characteristics during curing.

Preferably the polyetherimide coating according to the first aspect of the invention comprises a second diamine such as monoaromatic diamines, aliphatic polyetherdiamines, aromatic polyetherdiamines aliphatic Jeff diamines, aromatic Jeff diamines, diamino terminated polysiloxanes or metal salts of aromatic diamines. A Jeff amine may be defined as a polyether compound which contains at least one primary amino group attached to the terminus of a polyether backbone, wherein the polyether backbone is based either on propylene oxide (PO), ethylene oxide (EO), or mixed EO/PO.

Polyetherimides comprising monoaromatic diamines such as diaminobenzoic acid (DABA), 2,6-diaminopyridine (DAPY) or 3,5-diaminophenol (DAPH) should improve polyetherimide coating adhesion since the aforementioned diamines provide carboxylic acid, pyridine and hydroxyl functional groups respectively, which can interact with the carbon steel substrate through acid-base interactions and/or H-bonding. Alternatively polyetherimides comprising aliphatic Jeff amines, aromatic Jeff amines, diamino terminated polysiloxanes such as aminopropyl terminated polydimethylsiloxane (PDAS) or metal salts of aromatic diamines such as divalent amino benzoic acid metal salt (DABM) exhibit improved formability due the presence of flexible ether linkages along the polymeric backbone. Particularly suitable Jeff amines include O,O′-Bis(2-aminopropyl) polypropylene glycol-block-polyethylene glycol-block-polypropylene glycol (J1), 4,7,10-trioxa-1,13-tridecanediamine (J2), Poly(propylene glycol) bis(2-aminopropyl ether having a molecular weight 230 (J3), Poly(propylene glycol) bis(2-aminopropyl ether having a molecular weight 400 (J4) and 1,2-bis(2-aminoethoxyethane) (J5). In addition, the formability of the polyetherimide coating can be increased further by selecting monomers having an increased number of ether groups and/or by selecting aliphatic diamines. The selection of aliphatic diamines reduces the glass transition temperature (T_{g}) of the polyamic acid intermediate, which enables lower temperatures to be used when curing said polyamic acid intermediate to form the polyetherimide.

Preferably the polyetherimide coating comprises a dianhydride having the chemical formula:

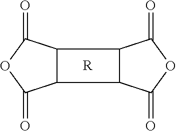

Advantageously, dianhydrides having the above chemical formula comprise aromatic groups which improve the corrosion resistance and the mechanical properties of the polyetherimide. Dianhydrides used in accordance with the invention include 3,3′,4,4′-Biphenyltetracarboxylic dianhydride (BPDA), pyrometallic dianhydride (PMDA), Benzophenone tetracarboxylic dianhydride (BPTA), 4,4′-Bisphenol A dianhydride (BPADA), 4,4′ Oxydiphthalic Anhydride (ODPA) and 4,4′-(hexafluoro-isopropylidene) diphthalic anhydride (FDA). Dianhydrides such as BPDA, BPTA and BPADA contain very rigid biphenyl structures that improve the corrosion resistance and the mechanical properties of the polyetherimide. The copolymerisation of a dianhydride such as ODPA should result in more adhesive and formable polyetherimides due to the presence of ether groups along the polymeric backbone, whereas the copolymerisation of FDA should lead to a polyetherimide exhibiting improved adhesive properties due to the presence of highly polar fluorine groups which can interact strongly with the substrate.

Preferably the polyetherimide coating comprises an end capper. The end capper is mixed with the polyamic acid intermediate to form an end capper/polyamic acid intermediate mixture, which is applied on the substrate and subsequently cured. Advantageously, the incorporation of an end capper reduces porosity within the resulting polyetherimide causing an improvement in the corrosion resistance of the polyetherimide coating.

Preferably the end capper is an aryl amine derivative or an amine terminated silane/siloxane, which should improve the adhesion between the carbon steel substrate and the polyetherimide coating if the aryl amine derivative comprises carboxylic acids, esters, amines or hydroxyl functional groups. Other aryl amine derivatives comprise phenol, acetylene or silanes. The use of an amine terminated silane/siloxane can also increase the formability of the polyetherimide coating due to the presence of a flexible polymeric chain. Suitable end cappers include 3-Aminopropyltriethoxysilane (APTES), 3-aminobenzoic acid (3-ABA), 3-amino phenol (3-ABP) and alkyl 3-aminobenzoate (3-ABE).

Preferably the organic solvent comprises N-Methylpyrrolidone (NMP), Dimethylacetamide (DMAC), Dimethylformamide (DMF) and/or ethanol. The use of NMP, DMAC and DMF, which are basic solvents avoids the formation of polyamic acid intermediate by-products and also accelerates the kinetics of the imidisation reaction upon curing. The use of ethanol is preferable since it is more environmentally friendly and readily available.

Preferably the polyamic acid intermediate is cured using a multistep heat treatment in the range of 100° C. to 300° C. Subjecting the polyamic acid intermediate to the heat treatment results in the formation of the corresponding polyetherimide having improved toughness, hardness and corrosion resistance. Improvement in formability is observed for polyetherimide coatings prepared using the multi-step approach, wherein the polyamic acid intermediate is subjected to a heat treatment for 15 minutes at 100° C., 15 minutes at 150° C. and 10 minutes at 300° C. When Infrared heating is used, the polyamic acid intermediate is subjected to a heat treatment between 1 second and 10 minutes at 100° C., between 1 second and 10 minutes at 150° C. and between 1 second and 10 minutes at 300° C. The use of the multi-step approach is also beneficial since it does not introduce sudden thermal shock and stresses in the polymer upon curing, it avoids the formation of solvent bubbles and it avoids the entrapment of solvents inside the polymer chains.

It is also possible to carry out the heat treatment in a single step at a temperature in the range of 200° C. to 300° C. The use of the single step approach offers a cost benefit to the manufacturer since the heat treatment is for a period between 30 seconds and 15 minutes and preferably for a period no longer than 5 minutes.

Preferably the polyetherimide coating has a dry film thickness in the range of 1 μm to 20 μm, more preferably a dry film thickness of 1 μm to 10 μm and even more preferably a dry film thickness of 2 μm to 6 μm. Advantageously, thicker coatings exhibit an increase in corrosion resistance whereas thinner coatings improve coating performance with respect to weldability and formability. Preferably, the coatings are applied on the substrate by roller coating, dipping or spraying and are dried and/or cured using convection heating, induction heating, direct heating or infrared heating.

The excellent adhesion and corrosion resistance properties that characterise the polyetherimides of the present invention means that it is no longer necessary to pre-treat carbon steel substrates, the coatings can instead be applied directly on a bare carbon steel substrate. Excellent adhesion and corrosion resistance is achieved through a combination of 1) Hydrogen bonding between oxide and hydroxide groups on the carbon steel surface and polar groups of the polyetherimide and 2) acid-base interactions between protonated nitrogen atoms of the polyetherimide and iron cations on the carbon steel surface.

Preferably the carbon steel substrate is pre-treated with a metallic and/or organic coating to enhance the overall corrosion protection. The polyetherimide coating exhibits improved corrosion resistance on carbon steel surfaces pre-treated with coatings of nickel, zinc, zinc oxide and alloys of zinc and aluminium. Improved adhesion is also observed on metallic surfaces pre-coated with silane or zirconium since strong covalent bonds are formed between the polyetherimide and the pre-treated substrate surface. Polar groups of the polyetherimide are also able to hydrogen bond with hydroxyl groups on the pre-treated surface.

Preferably the polyetherimide coating is provided on a hot-rolled carbon steel substrate or a cold-rolled carbon steel substrate. Advantageously, the adhesion properties of the coating are improved since the polyetherimide can interact favourably through acid-base interactions and/or H-bonding. Carbon steel bodies include rebars, wires, sheets and plates.

Preferably there is provided a method of preparing a polyetherimide coating on a carbon steel substrate wherein the polyamic acid intermediate produced according to the first or second aspect of the invention and the embodiments hereinabove is mixed with an aliphatic polyamic acid and/or an aromatic polyamic acid and an additive to form a polyamic acid mixture. Advantageously, the polyamic acid mixture is applied on the substrate and cured to form a polyetherimide blend. The polyetherimide blend can be tailored to improve corrosion resistance, adhesion and formability. The additive comprises wetting agents, antioxidising agents and buffering agents such as PO₄⁻³, SiO₃⁻ and MoO₄⁻.

In a third aspect of the invention there is provided the polyamic acid intermediate of the first aspect of the invention or the second aspect of the invention. The polyamic acid intermediate according to the first aspect of the invention comprises a dianhydride, a first diamine and a second diamine, whereas the polyamic acid intermediate according to the second aspect of the invention comprises a dianhydride and a second diamine. The polyamic acid intermediate is manufactured according to the first aspect of the invention or the second aspect of the invention and the preferred embodiments disclosed hereinabove are similarly applicable to the polyamic acid intermediate. The polyamic acid intermediate can be used as a precursor for producing adhesives, matrix resins for composites, fibres, mouldings and the like.

In a fourth aspect of the invention there is provided a method of preparing a polyetherimide fibre wherein the polyamic acid intermediate of the third aspect of the invention is formed into a fibre and then cured. The polyamic acid solution is preferably filtered and degassed at 100° C. prior to spinning the polyamic acid intermediate into the form of a fibre. Such fibres can be prepared by dry-jet-wet-spinning with an air gap of approximately 20 mm. The filtered and degassed polyamic acid intermediate is preferably extruded through a spinneret with up to six orifices measuring approximately 0.08 mm in diameter. The spun fibres are preferably passed through a first washing bath comprising water and alcohol to provide fibres of uniform microstructure. The fibres then enter a second washing bath comprising ethanol. The spun fibres can then be cured at a temperature of 300° C. or less.

In a fifth aspect of the invention there is provided a polyetherimide coated substrate produced according to the method of the first aspect of the invention or the second aspect of the invention. The polyetherimide coating according to the first aspect of the invention comprises a dianhydride, a first diamine and a second diamine, whereas the polyetherimide coating according to the second aspect of the invention comprises a dianhydride and a second diamine. The preferred embodiments disclosed hereinabove are similarly applicable to the polyetherimide coated substrate.

Embodiments of the present invention will now be described by way of example. These examples are intended to enable those skilled in the art to practice the invention and do not in anyway limit the scope of the invention as defined by the claims.

Scheme 1 shows the general method for preparing a polyamic acid intermediate and a polyetherimide thereof.

FIG. 1 shows differential scanning calorimetry (DSC) graphs showing Tg and the amorphous nature of the polyetherimides according to examples 1, 2, 3, 4 and 6. The glass transitions temperatures for these polyetherimide coatings can be seen in Table 1.

Table 1 shows results relating to molecular weight, PDI, Tg, relative viscocity (ηr), temperature at 10% weight loss and Salt spray tests (SST).

Table 2 shows the results of impendence studies which reflect the corrosion resistance of polyetherimides.

Electrochemical impedance spectroscopy (EIS) experiments were carried out using simulated saline medium (3.5% NaCl solution: pH: 6.7) to evaluate the charge-transfer resistance (R_{ct}), also called the polarisation resistance (Rₚ). and the total coating capacitance Cc, which is a measure of water diffusion through a coating. Cc is equal to the sum of the film capacitance (C_{f}) and the double-layer capacitance (C_{dl}) and corrosion rate which is inversely proportional to Rₚ. Modulus of impedance IZI gives an indication of barrier properties of the film. A high IZI value corresponds with improved barrier properties. The experimental set up comprises the working electrode (coated steel substrates whose characteristics to be evaluated), reference electrode (calomel) and counter electrode (Ni). The working electrode area was selected by using a Teflon holder that exposed a disk of area 12 cm². Impedance measurements were performed using an EG&G PARC 273A potentiostat and a Solartron 1255 frequency response analyzer controlled by a microcomputer running ZPLOT software (Scribner Associates, Charlottesville, Va.). Impedance values were determined at five discrete frequencies per decade over the range 10.0 mHz to 65 kHz. The experimental data thus obtained were fitted with Randles' equivalent circuit model using ZSIM/CNLS software (Scribner Associates). The Randles' equivalent circuit is not the only possible representation but has been frequently employed to represent modified electrochemical interfaces, and in the present case provides an excellent fit of the data.

Poly(amic)acid (PAA) viscosity was measured with a SCHOTT Viscometer, with Visco System AVS 470, at 25° C. The film can be cast if the inherent relative viscosity is between 0.2 dL/g and 5 dL/g, preferably between 0.4 dL/g and 2 dL/g. Moreover, high viscosity can be associated with high molecular weight. Thus viscosity gives an indication of molecular weight and the conversion rate of polymerization.

Gas Phase Chromatography (GPC) analyses were performed at 60° C. with 0.5 mL/min flow in a LF 804 column from shodex, filled with LiBr solution in NMP (5 mmol/L). The PAA concentration of analyzed samples was 0.8 mg/mL.

Thermogravimetric analyses (TGA) were conducted using a Perkin Elmer pyris diamond DMA, Differential scanning calorimetry (DSC) was performed on a Perkin Elmer Pyris sapphire DSC and Dynamic mechanical thermal analyses (DMTA) were carried out on a Perkin Elmer pyris diamond DMA. Scans were recorded with the following settings:
TGA: heating rate: 10° C./min
   Temperature range: 25° C. to 600° C.
DSC: heating/cooling rate: 20° C./min
   Temperature range: 25° C. to 450° C.
   Program: 1) heating to 450° C. 2) cooling to 25° C. 3) heating to 450° C.
DMTA: heating rate of 2° C./min.
   Temperature range: 25° C. to 300° C.
   Vibration frequency: 1 Hz

All analyses were performed under nitrogen flow on free standing polyetherimide films peeled off glass plates. The thermal stability of the polymer is evaluated by TGA. Temperature for 5% wt and 10% wt loss are standard measures of the polymer stability. The glass transition temperature (Tg) can be obtained with DSC and more accurately with DMTA. DMTA is also used to determine the mechanical properties of the polyetherimide coatings over a large temperature range.

The Salt spray test (ASTM B117 standard) is used to measure the corrosion resistance of coated and uncoated metallic specimens, when exposed to a salt spray at elevated temperature. Polyetherimide coated substrates were placed in an enclosed chamber at 35° C. and exposed to a continuous indirect spray (fogging) of 5% salt solution (pH 6.5 to 7.2), which falls-out on to the coated substrate at a rate of 1.0 to 2.0 ml/80 cm²/hour. The fogging of 5% salt solution is at the specified rate and the fog collection rate is determined by placing a minimum of two 80 sq. cm. funnels inserted into measuring cylinders graduated in ml. inside the chamber. This climate is maintained under constant steady state conditions. The samples are placed at a 15-30 degree angle from vertical. The test duration is variable. The sample size is 76×127×0.8 mm, are cleaned, weighed, and placed in the chamber in the proximity of the collector funnels. After exposure the panels are critically observed periodically for blisters, delaminations and red rust.

### EXAMPLE 1

### Synthesis of PI 16

A 100 mL one neck flask equipped with a nitrogen inlet is heated for 10 minutes with a heat gun under nitrogen flow to remove water and oxygen from the flask. The flask is then charged with (3.5 mmol, 1.044 g) 1,3-bis(4-aminophenoxy)benzene (98%), (1.5 mmole, 0.165 g) of m-phenylenediamine (99%) and 23.9 g of NMP. (5 mmole, 1.5166 g) of 4,4′-Biphthalic Anhydride (97%) is then added to the solution and the solution is stirred under N₂ for 8 to 16 hrs to form a polyamic acid intermediate (PAA 16). PAA 16 is then applied directly on the steel substrate and cured in an oven at 250° C. for 5 min to form a PI 16 polyetherimide coated steel product. The storage modulus, which is a measure polymer strength, was recorded at 4.4 (Gpa) for the PI 16 polyetherimide.

### EXAMPLE 2

### Synthesis of PI 16.1

A 100 mL one neck flask equipped with a nitrogen inlet is heated for 10 minutes with a heat gun under nitrogen flow to remove water and oxygen from the flask. The flask is then charged with (3.5 mmol, 1.044 g) 1,3-bis(4-aminophenoxy)benzene (98%), (1.0 mmol, 0.165 g) of m-phenylenediamine (99%), (0.5 mmole, 0.076 gm) 3,5-diaminobenzoic acid and 23.9 g of NMP. (5 mmole, 1.5166 g) of 4,4′-Biphthalic Anhydride (97%) is then added to the solution and the solution is stirred under N₂ for 8 to 16 hrs to form a polyamic acid intermediate (PAA 16.1). PAA 16.1 is then applied directly on the steel substrate and cured in an oven at 250° C. for 5 min to form a PI 16.1 polyetherimide coated steel product. The storage modulus of the PI 16.1 polyetherimide was 4.9 (Gpa).

### EXAMPLE 3

### Synthesis of PI 16.2

A 150 mL one neck flask equipped with a nitrogen inlet is heated for 10 minutes with a heat gun under nitrogen flow to remove water and oxygen from the flask. The flask is then charged with (12 mmol, 3.58 g) 1,3-bis(4-aminophenoxy)benzene (98%), (5 mmole, 0.55 g) of m-phenylenediamine (99%), (3 mmole, 0.334 gm) 1,5-diaminopyridine and 100 g of NMP. (20 mmole, 6.0644 g) of 4,4′-Biphthalic Anhydride (97%) is then added to the solution and the solution is stirred under N₂ for 8 to 16 hrs to form a polyamic acid intermediate (PAA 16.2). PAA 16.2 is then applied directly on the steel substrate and cured in an oven at 250° C. for 5 min to form a PI 16.2 polyetherimide coated steel product. The storage modulus of the PI 16.2 polyetherimide was 8.7 (Gpa).

### EXAMPLE 4

### Synthesis of PI 16.3

A 100 mL one neck flask equipped with a nitrogen inlet is heated for 10 minutes with a heat gun under nitrogen flow to remove water and oxygen from the flask. The flask is then charged with (3.5 mmol, 1.044 g) 1,3-bis(4-aminophenoxy)benzene (98%), (1.25 mmole, 0.135 g) of m-phenylenediamine (99%), (0.25 mmole, 0.238 gm) polydimethylsiloxane, aminopropyl terminated and 23.9 g of NMP. (5 mmole, 1.5166 g) of 4,4′-Biphthalic Anhydride (97%) is then added to the solution and the solution is stirred under N₂ for 8 to 16 hrs to form a polyamic acid intermediate (PAA 16.3). PAA 16.3 is then applied directly on the steel substrate and cured in an oven at 250° C. for 5 min to form a PI 16.3 polyetherimide coated steel product.

### EXAMPLE 5

### Synthesis of PI 16.4

Synthesis of Mn-diaminobenzoic salt (DABM): 2:1 mole ration of amino benzoic acid and MnO₂ was mixed in 100 ml of water and heated at 60° C. for 4 hrs. This mixture was then filtered and dried in the oven for 24 hrs.

A 100 mL one neck flask equipped with a nitrogen inlet is heated for 10 minutes with a heat gun under nitrogen flow to remove water and oxygen from the flask. The flask is then charged with (3.5 mmol, 1.044 g) 1,3-bis(4-aminophenoxy)benzene (98%), (1.3 mmole, 0.142 g) of m-phenylenediamine (99%), (0.2 mmole, 0.066 gm) DABM and 23.9 g of NMP. (5 mmole, 1.5166 g) of 4,4′-Biphthalic Anhydride (97%), is then added to the solution and the solution is stirred under N₂ for 8 to 16 hrs to form a polyamic acid intermediate (PAA 16.4). PAA 16.4 is then applied directly on the steel substrate and cured in an oven at 250° C. for 5 min to form a PI 16.4 polyetherimide coated steel product.

### EXAMPLE 6

### Synthesis of PI 16.5

A 100 mL one neck flask equipped with a nitrogen inlet is heated for 10 minutes with a heat gun under nitrogen flow to remove water and oxygen from the flask. The flask is then charged with (5 mmole, 3 gm) O,O′-Bis(2-aminopropyl) polypropylene glycol-block-polyethylene glycol-block-polypropylene glycol and 23.9 g of NMP. (5 mmol, 1.5166 g) of 4,4′-Biphthalic Anhydride (97%), is then added to the solution and the solution is stirred under N₂ for 8 to 16 hrs to form a polyamic acid intermediate (PAA 16.5). PAA 16.5 is then applied directly on the steel substrate and cured in an oven at 250° C. for 5 min to form a PI 16.5 polyetherimide coated steel product. The storage modulus of the PI 16.5 polyetherimide was 4.4 (Gpa).

### EXAMPLE 7

### Synthesis of PI 16 EC-1

A 100 mL one neck flask equipped with a nitrogen inlet is heated for 10 minutes with a heat gun under nitrogen flow to remove water and oxygen from the flask. The flask is then charged with (1.8 mmol, 0.557 g) 1,3-bis(4-aminophenoxy)benzene (98%), (0.777 mmole, 0.85 g) of m-phenylenediamine (99%) and 23.9 g of NMP. (3.29 mmole, 0.99 g) of 4,4′-Biphthalic Anhydride (97%) is then added to the solution and the solution is stirred under N₂ for 8 to 16 hrs. Thereafter, (0.71 mmole, 0.156 g) of aminopropyl triethoxysilane end-capper is added to the solution to form a mixture, which is stirred overnight. This mixture is then applied directly on the steel substrate and cured in an oven at 250° C. for 5 min to form a PI 16 EC-1 polyetherimide coated steel product.

### EXAMPLE 8

### Synthesis of PI 16 EC-2

A 100 mL one neck flask equipped with a nitrogen inlet is heated for 10 minutes with a heat gun under nitrogen flow to remove water and oxygen from the flask. The flask is then charged with (1.8 mmol, 0.557 g) 1,3-bis(4-aminophenoxy)benzene (98%), (0.777 mmole, 0.85 g) of m-phenylenediamine (99%) and 23.9 g of NMP. (3.29 mmole, 0.99 g) of 4,4′-Biphthalic Anhydride (97%) is then added to the solution and the solution is stirred under N₂ for 8 to 16 hrs. Thereafter, (0.71 mmole, 0.098 g) of 4-aminobenzoic acid end-capper was added to the solution to form a mixture, which is stirred overnight. This mixture is then applied directly on the steel substrate and cured in an oven at 250° C. for 5 min to form a PI 16 EC-2 polyetherimide coated steel product.

### EXAMPLE 9

### Synthesis of PI 16 EC-3

A 100 mL one neck flask equipped with a nitrogen inlet is heated for 10 minutes with a heat gun under nitrogen flow to remove water and oxygen from the flask. The flask is then charged with (1.8 mmol, 0.557 g) 1,3-bis(4-aminophenoxy)benzene (98%), (0.777 mmole, 0.85 g) of m-phenylenediamine (99%) and 23.9 g of NMP. (3.29 mmole, 0.99 g) of 4,4′-Biphthalic Anhydride (97%) is then added to the solution and the solution is stirred under N₂ for 8 to 16 hrs. Thereafter, (0.71 mmole, 0.107 g) of methyl-3 aminobenzoate end-capper is added to the solution to form a mixture, which is stirred overnight. This mixture is then applied directly on the steel substrate and cured in an oven at 250° C. for 5 min to form a PI 16 EC-3 polyetherimide coated steel product.

### EXAMPLE 10

### Synthesis of PI 16 EC-4

A 100 mL one neck flask equipped with a nitrogen inlet is heated for 10 minutes with a heat gun under nitrogen flow to remove water and oxygen from the flask. The flask is then charged with (1.8 mmol, 0.557 g) 1,3-bis(4-aminophenoxy)benzene (98%), (0.777 mmole, 0.85 g) of m-phenylenediamine (99%) and 23.9 g of NMP. (3.29 mmole, 0.99 g) of 4,4′-Biphthalic Anhydride (97%) is then added to the solution and the solution is stirred under N₂ for 8 to 16 hrs. Thereafter, (0.71 mmol, 0.076 g) of 3-aminophenol end-capper is added to the solution to form a mixture, which is stirred overnight. This mixture is then applied directly on the steel substrate and cured in an oven at 250° C. for 5 min to form a PI 16 EC-4 polyetherimide coated steel product.

### EXAMPLE 11

### Synthesis of BJ2

A 100 mL one neck flask equipped with a nitrogen inlet is heated for 10 minutes with a heat gun under nitrogen flow to remove water and oxygen from the flask. The flask is then charged with (10 mmol, 2.203 g) of 4,7,10-trioxa-1,13-tridecanediamine (J2) and 40 g of NMP to form a solution which was stirred at 80° C. for 5 min. (10 mmole, 3.032 g) of 4,4′-Biphthalic Anhydride (97%) is added to the solution and stirring is continued for a further 2 hrs at 80° C. The temperature is then increased to 120° C. and stirred for an additional 2 hrs before the solution is applied directly on the steel substrate and cured in the oven at 250° C. for 5 min to form a BJ2 polyetherimide coated steel product.

### EXAMPLE 12

### Synthesis of BJ2.1

A 100 mL one neck flask equipped with a nitrogen inlet is heated for 10 minutes with a heat gun under nitrogen flow to remove water and oxygen from the flask. The flask is then charged with (10 mmole, 3.032 g) of 4,4′-Biphthalic Anhydride (97%) and 40 gm of dry ethanol to form a solution. The flask is connected to a condenser and the solution is heated and stirred at reflux for 1 hr. (10 mmol, 2.203 g) of 4,7,10-trioxa-1,13-tridecanediamine (J2) is then added to the solution which is then stirred at 60° C. to 80° C. for 4 hrs before the solution is applied directly on the steel substrate and cured in the oven at 250° C. for 5 min to form a BJ2.1 polyetherimide coated steel product.

### EXAMPLE 13

### Synthesis of BJ3

A 100 mL one neck flask equipped with a nitrogen inlet is heated for 10 minutes with a heat gun under nitrogen flow to remove water and oxygen from the flask. The flask is then charged with (10 mmole, 3.032 g) of 4,4′-Biphthalic Anhydride (97%) and 40 gm of dry ethanol to form a solution. The flask is connected to a condenser and the solution is heated and stirred at reflux for 1 hr. (10 mmol, 2.3 g) of Poly(propylene glycol) bis(2-aminopropyl ether having a molecular weight 230 (J3) is then added to the solution which is then stirred at 60° C. to 80° C. for 4 hrs before the solution is applied directly on the steel substrate and cured in the oven at 250° C. for 5 min to form a BJ3 polyetherimide coated steel product.

### EXAMPLE 14

### Synthesis of BJ4

A 100 mL one neck flask equipped with a nitrogen inlet is heated for 10 minutes with a heat gun under nitrogen flow to remove water and oxygen from the flask. The flask is then charged with (10 mmole, 3.032 g) of 4,4′-Biphthalic Anhydride (97%) and 40 gm of dry ethanol to form a solution. The flask is connected to a condenser and the solution is heated and stirred at reflux for 1 hr. (10 mmol, 4 g) of Poly(propylene glycol) bis(2-aminopropyl) ether having a molecular weight of 400 (J4) is then added to the solution which is stirred at 60° C. to 80° C. for 4 hrs. The solution is then applied directly on the steel substrate and cured in the oven at 250° C. for 5 min to form a BJ4 polyetherimide coated steel product.

### EXAMPLE 15

### Synthesis of BJ5

A 100 mL one neck flask equipped with a nitrogen inlet is heated for 10 minutes with a heat gun under nitrogen flow to remove water and oxygen from the flask. The flask is then charged with (10 mmole, 3.032 g) of 4,4′-Biphthalic Anhydride (97%) and 40 gm of dry ethanol to form a solution. The flask is connected to a condenser and the solution is heated and stirred at reflux for 1 hr. (10 mmol, 1.48 g) of and 1,2-bis(2-aminoethoxyethane) (J5) having mol. Wt 148.20 is then added to the solution which is then stirred at 60° C. to 80° C. for 4 hrs before the solution is applied directly on the steel substrate and cured in the oven at 250° C. for 5 min to form a BJ5 polyetherimide coated steel product.

### EXAMPLE 16

### Synthesis of BJ5.2

A 100 mL one neck flask equipped with a nitrogen inlet is heated for 10 minutes with a heat gun under nitrogen flow to remove water and oxygen from the flask. The flask is then charged with (7 mmol, 1.0374 g) of 1,2-bis(2-aminoethoxyethane) (J5) and (3 mmole, 0.3244 gm) of m-phenylenediamine (99%) and 40 g of NMP to form a solution which was stirred at 80° C. for 5 min. (10 mmole, 3.032 g) of 4,4′-Biphthalic Anhydride (97%) is added to the solution and stirring is continued for a further 2 hrs at 80° C. The temperature is then increased to 120° C. and stirred for an additional 2 hrs before the solution is applied directly on the steel substrate and cured in the oven at 250° C. for 5 min to form a BJ5.2 polyetherimide coated steel product.

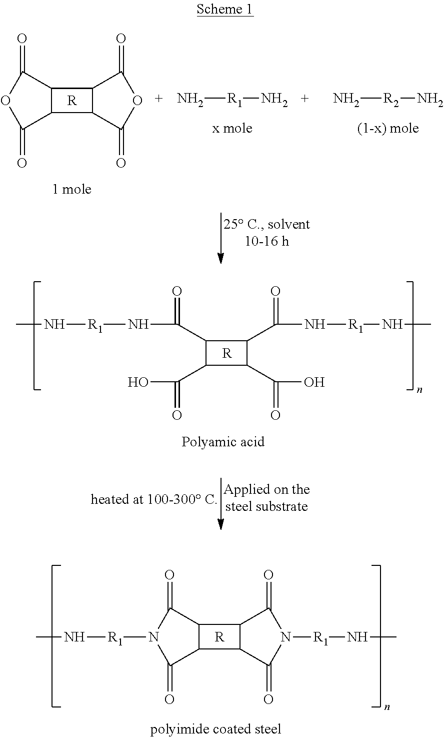

TABLE 1

| | | | | | | Td (10% wt. | |
|---|---|---|---|---|---|---|---|
| | Amine | | | ηᵣ | Tg | loss) | Hrs in |
| Example | components | Mol. wt | PDI | (gm/dl) | (° C.) | ° C. | SST |
| 1 | M1, MPA | 132,530 | 3.40 | 0.62 | 205 | 550 | 336^{(d)} |
| 2 | M1, MPA, DABA | 59,000 | 1.58 | 0.82 | 195 | 515 | 250^{(c)} |
| 3 | M1, MPA, DAPY | 38,000 | 1.88 | 0.57 | 197 | 526 | 350^{(b)} |
| 4 | M1, MPA, PDAS | 148,420 | 2.54 | 0.75 | 225 | 540 | 300^{(a)} |
| 6 | M1, MPA, DAPH | 209,939 | 3.18 | 0.28 | 204 | 358 | 400^{(b)} |
| 11 | J2 | 2074 | — | 0.3 | 55 | 435 | 48^{(a)} |
| 13 | J3 | 2636 | 1.8 | 0.2 | 95 | 427 | 250^{(a)} |
| 14 | J4 | — | — | 0.25 | 60 | 367 | 48^{(a)} |
| 15 | J5 | 66000 | 2.6 | 0.28 | 120 | 413 | 300^{(a)} |
| 16 | MPA, J5 | 120000 | 1.7 | 0.36 | 155 | 475 | 400^{(b)} |

^{(a)}<10% surface delamination,
^{(b)}<10% red rust,
^{(c)}<10% blisters,
^{(d)}<10% surface delamination and red rust.

TABLE 2

| | Time | IzI, (Ohm at | Cc | Rp |
|---|---|---|---|---|
| Example | (hrs) | Logf: 0.16 Hz) | (μF/cm²) | (ohm · cm²) |
| 1 | 1 | 4.47 × 10⁷ | 0.0017 | 2.26 × 10⁸ |
| | 24 | 4.68 × 10⁷ | 0.0017 | 2.87 × 10⁷ |
| | 182 | 2.43 × 10⁶ | 0.0341 | 2.47 × 10⁶ |
| | 336 | 3.66 × 10⁵ | 0.225 | 3.66 × 10⁵ |
| 2 | 1 | 1.2 × 10⁷ | 0.0069 | 3.11 × 10⁷ |
| | 24 | 1.61 × 10⁴ | 5.1 | 1.11 × 10⁴ |
| | 182 | — | — | — |
| | 336 | 1.38 × 10³ | 60 | 887 |
| 3 | 1 | 1.89 × 10⁵ | 0.0022 | 2.8 × 10⁶ |
| | 24 | 4.42 × 10⁴ | 0.0118 | 6.82 × 10³ |
| | 182 | 1.3 × 10⁴ | 0.0444 | 1.29 × 10⁵ |
| | 336 | 9.88 × 10³ | 0.12 | 5.55 × 10³ |
| 4 | 1 | 2.6 × 10⁷ | 0.02 | 2.28. × 10⁵ |
| | 24 | 1.2 × 10⁷ | 0.07 | 1.26 × 10⁷ |
| | 182 | 2.2 × 10⁶ | 0.12 | 2.1 × 10⁵ |
| | 336 | 0.037 × 10⁶ | 2.22 | 0.12 × 10⁶ |
| 6 | 1 | 4.74 × 10⁵ | 0.21 | 3.22 × 10⁶ |
| | 24 | 2.07 × 10⁴ | 4 | 6.18 × 10³ |
| | 182 | 17.73 × 10³ | 4.7 | 19.335 × 10³ |
| | 336 | 1.37 × 10³ | 71.1 | 1.72 × 10³ |
| 13 | 24 | 7.9 × 10³ | 12.6 | 9.4 × 10³ |
| | 96 | 3.5 × 10³ | 28.5 | 3.8 × 10³ |
| | 192 | 1.9 × 10³ | 52.6 | 2.2 × 10³ |
| | 400 | 1.7 × 10³ | 58.8 | 2.1 × 10³ |
| 16 | 24 | 2.81 × 10⁶ | 0.03 | 1 × 10⁷ |
| | 96 | 2.28 × 10⁵ | 0.4 | 7.49 × 10⁵ |
| | 192 | 8 × 10³ | 12.5 | 2.8 × 10⁴ |
| | 400 | 2.2 × 10⁴ | 4.5 | 5.7 × 10⁴ |

1. A method of preparing a polyetherimide coating on a carbon steel substrate, which comprises the steps of:
i. providing an organic solvent;
ii. providing a dianhydride;
iii. providing a first diamine that is a monoaromatic diamine;
iv. providing a second diamine;
v. placing the organic solvent, the dianhydride, the first diamine and the second diamine in a reaction vessel to form a reaction mixture;
vi. stirring the reaction mixture under inert conditions to form a polyamic acid intermediate;
vii. applying the polyamic acid intermediate on the carbon steel substrate;
viii. curing the polyamic acid intermediate to form the polyetherimide coating.

2. A method of preparing a polyetherimide coating on a carbon steel substrate, which comprises the steps of:
i. providing an organic solvent;
ii. providing a dianhydride;
iii. providing a diamine;
iv. placing the organic solvent, the dianhydride and the diamine in a reaction vessel to form a reaction mixture;
v. stirring the reaction mixture under inert conditions to form a polyamic acid intermediate;
vi. applying the polyamic acid intermediate on the substrate;
vii. curing the polyamic acid intermediate to form a polyetherimide coating.

3. The method of preparing the polyetherimide coating on a carbon steel substrate according to claim 2, wherein the diamine is a polyether compound that contains at least one primary amino group attached to a terminus of a polyether backbone, wherein the polyether backbone is based either on propylene oxide, ethylene oxide or mixed propylene oxide/ethylene oxide.

4. The method of preparing the polyetherimide coating on a carbon steel substrate according to claim 1, wherein the polyetherimide coating comprises a third diamine that is a polyetherdiamine.

5. The method of preparing the polyetherimide coating on a carbon steel substrate according to claim 2, wherein the polyetherimide coating comprises an additional diamine that is a polyetherdiamine.

6. The method of preparing the polyetherimide coating on a carbon steel substrate according to claim 1, wherein the first diamine is a substituted monoaromatic diamine.

7. The method of preparing the polyetherimide coating on a carbon steel substrate according to claim 6, wherein the first diamine is a meta substituted monoaromatic diamine, wherein the meta substituted monoaromatic diamine is mphenylenediamine.

8. The method of preparing the polyetherimide coating on a carbon steel substrate according to claim 1, wherein the second diamine comprises at least one member selected from the group consisting of monoaromatic diamines, aliphatic polyetherdiamines, aromatic polyetherdiamines, aliphatic diamine which is a polyether compound that contains at least one primary amino group attached to a terminus of a polyether backbone, wherein the polyether backbone is based either on propylene oxide, ethylene oxide or mixed propylene oxide/ethylene oxide, aromatic diamines which are a polyether compound that contains at least one primary amino group attached to a terminus of a polyether backbone, wherein the polyether backbone is based either on propylene oxide, ethylene oxide or mixed propylene oxide/ethylene oxide, diamino terminated polysiloxanes and metallic salts of aromatic diamines.

9. The method of preparing the polyetherimide coating on a carbon steel substrate according to claim 1, wherein an end capper is mixed with the polyamic acid intermediate to form an end capper/polyamic acid intermediate mixture, which is applied on the substrate and subsequently cured.

10. The method of preparing the polyetherimide coating on a carbon steel substrate according to claim 9, wherein the end capper is an aryl amine derivative or an amine terminated silane or siloxane.

11. The method of preparing the polyetherimide coating on a carbon steel substrate according to claim 1, wherein the polyamic acid intermediate is cured using a multi-step heat treatment in the range of 100° C. to 300° C.

12. The method of preparing the polyetherimide coating on a carbon steel substrate according to claim 1, wherein the polyetherimide coating has a dry film thickness after curing in the range of 1 μm to 20 μm.

13. The method of preparing the polyetherimide coating on a carbon steel substrate according to claim 1, wherein the carbon steel substrate is pre-treated with a metallic coating and/or organic coating.

14. The method of preparing the polyetherimide coating on a carbon steel substrate according to claim 1, wherein the carbon steel substrate is a rebar, wire, sheet or plate.

15. The method of preparing the polyetherimide coating on a carbon steel substrate wherein the polyamic acid intermediate produced according to the method of claim 1 is mixed with an aliphatic polyamic acid and/or an aromatic polyamic acid and an additive to form a polyamic acid mixture.

16. The polyamic acid intermediate made according to a method comprising the steps of:
i. providing an organic solvent;
ii. providing a dianhydride;
iii. providing a first diamine that is a monoaromatic diamine;
iv. providing a second diamine;
v. placing the organic solvent, the dianhydride, the first diamine and the second diamine in a reaction vessel to form a reaction mixture;
vi. stirring the reaction mixture under inert conditions to form a polyamic acid intermediate.

17. A method of preparing a polyetherimide fiber wherein the polyamic acid intermediate of claim 16 is formed into a fiber and then cured.

18. A polyetherimide coated metallic substrate obtainable by the process of claim 1.

19. A method of preparing a polyetherimide coating on a carbon steel substrate according to claim 1, wherein the polyetherimide coating comprises a third diamine that is an aromatic polyetherdiamine.

20. A method of preparing a polyetherimide coating on a carbon steel substrate according to claim 2, wherein the polyetherimide coating comprises an additional diamine that is an aromatic polyetherdiamine.

21. The method of preparing the polyetherimide coating on a carbon steel substrate according to claim 2, wherein an end capper is mixed with the polyamic acid intermediate to form an end capper/polyamic acid intermediate mixture, which is applied on the substrate and subsequently cured.

22. The polyamic acid intermediate made according to a method comprising the steps of:
i. providing an organic solvent;
ii. providing a dianhydride;
iii. providing a diamine;
iv. placing the organic solvent, the dianhydride and the diamine in a reaction vessel to form a reaction mixture;
v. stirring the reaction mixture under inert conditions to form a polyamic acid intermediate.

23. A method of preparing a polyetherimide fiber wherein the polyamic acid intermediate of claim 22 is formed into a fiber and then cured.

24. A polyetherimide coated metallic substrate obtainable by the process of claim 2.
